SEQUENCE LISTING (2 sequences)

SEQ ID NO: 1 - protein, 571 aa, Homo sapiens
GAPGAPGLQGAPGLQGMPGERGADGLPGPKGERGDAGPKGADGAPGAPGLQGMPGERGAAGLPGPKGERGDAGPKGAAGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGAAGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGERGDAGPKGADGAPGAPGLQGMPGERGAAGLPGPKGERGDAGPKGADGAPGKDGVRGLAGPIGPPGERGAAGLPGPKGERGDAGPKGADGAPGKDGVRGLAGPIGPPGPAGAPGAPGLQGMPGERGADGLPGPKGERGDAGPKGADGAPGKDGVRGLAGPPG

SEQ ID NO: 2 - protein, 571 aa, Homo sapiens
GAPGAPGLQGAPGLQGMPGERGAAGLPGPKGERGDAGPKGAAGAPGAPGLQGMPGERGAAGLPGPKGERGDAGPKGAAGAPGKDGVRGLAGPIGPPGERGAAGLPGPKGERGDAGPKGAAGAPGKDGVRGLAGPIGPPGPAGAPGAPGLQGMPGARGADGLPGPKGERGDAGPKGADGAPGKAGVRGLAGPPGAPGLQGAPGLQGMPGARGAAGLPGPKGARGDAGPKGAAGAPGAPGLQGMPGERGAAGLPGPKGERGDAGPKGAAGAPGKDGVRGLAGPIGPPGERGAAGLPGPKGERGDAGPKGADGAPGKDGVRGLAGPIGPPGPAGAPGAPGLQGMPGERGAAGLPGPKGERGDAGPKGADGAPGKDGVRGLAGPPGAPGLQGAPGLQGMPGERGAAGLPGPKGARGDAGPKGADGAPGAPGLQGMPGARGAAGLPGPKGERGDAGPKGADGAPGKDGVRGLAGPIGPPGERGAAGLPGPKGERGDAGPKGAAGAPGKAGVRGLAGPIGPPGPAGAPGAPGLQGMPGERGAAGLPGPKGERGDAGPKGADGAPGKDGVRGLAGPPG

(19) United States
(12) Patent Application Publication
(10) Pub. No.: US 2021/0106726 A1
(43) Pub. Date: Apr. 15, 2021

(54) Hydrogels

(71) Applicant: Fujifilm Manufacturing Europe B.V., Tilburg (NL)

(72) Inventors: Zahide Mumcuoglu, Tilburg (NL); Laura De Aragon, Tilburg (NL); Sebastianus Gerardus Johannes Maria Kluijtmans, Tilburg (NL)

(21) Appl. No.: 16/464,489

(22) PCT Filed: Nov. 23, 2017
(86) PCT No.: PCT/GB2017/053517

§ 371 Date: May 28, 2019

(30) Foreign Application Priority Data
Nov. 29, 2016 (GB) 1620204.6

(51) Int. Cl. A61L 27/52; A61L 27/26; A61L 27/02; A61L 27/36; A61L 27/54

(52) U.S. Cl. CPC A61L 27/52; A61L 27/26; A61L 27/025; A61L 2430/02; A61L 27/54; A61L 2300/414; A61L 2400/06; A61L 27/3608

(57) ABSTRACT

A hydrogel comprising: water; an alginate; a glucono-delta-lactone (GDL); and microparticles comprising an inorganic calcium compound and recombinant gelatin. The hydrogels may be used for bone repair and/or regeneration.

This invention relates to a hydrogel which may be used particularly for bone repair or bone regeneration.

WO 2013/077739 ('739) describes cements comprising calcium phosphate microparticles having a size of between 50 and 300 μm. The microparticles contain glucono-delta-lactone (GDL). When the cement is in a physiological environment, the GDL within the calcium phosphate microparticles hydrolyses and exits the microparticles, leaving behind voids in the microparticles. The porous microparticles so formed may then be used in bone regeneration. Yan et al in Mater Sci Eng C Biol Appl, 2016 describe injectable alginate/hydroxyapatite gel scaffolds combined with gelatin microspheres. The scaffolds are crosslinked using CaCO₃ and GDL. The microspheres described in Yan et al do not comprise an inorganic calcium compound. Instead in Yan et al the gelatin particles and inorganic compound particles are separate entities.

According to a first aspect of the present invention there is provided a hydrogel comprising:
water;
an alginate;
a glucono-delta-lactone (GDL); and
microparticles comprising an inorganic calcium compound and recombinant gelatin.

The term “comprising” as used in this specification is to be interpreted as specifying the presence of the stated parts, steps or components, but does not exclude the presence of one or more additional parts, steps or components.

Reference to an element by the indefinite article “a” or “an” does not exclude the possibility that more than one of the element(s) is present, unless the context clearly requires that there be one and only one of the elements. The indefinite article “a” or “an” thus usually means “at least one”.

Preferably the hydrogel is an injectable hydrogel.

The hydrogel typically comprises a network of hydrophilic polymer chains derived from the alginate. The hydrogel may be described as jelly-like and is very different from the cements described in '739. Typically the hydrogel ensnares the water, GDL and microparticles in three-dimensional gel network. The hydrogel preferably possesses a degree of flexibility which is very similar to natural tissue, due in part to its significant water content. Optionally the hydrogel is a thixotropic hydrogel, i.e. it becomes fluid when agitated and resolidifies when resting.

The hydrogel preferably comprises water in an amount of 65 to 97.5 wt %, more preferably 75 to 97 wt %, especially 85 to 97 wt %. The water content may be determined simply by drying a known weight of the hydrogel and determining the weight loss relative to the initial weight of the hydrogel.

The alginate is preferably alginic acid, a derivative of alginic acid, or a salt thereof, e.g. the potassium, calcium, ammonium, lithium or preferably sodium salt of alginic acid, a derivative of alginic acid or a mixture comprising two or more thereof. Alginates are available from commercial sources and are typically derived from seaweed. Alginates are biocompatible, have low toxicity and form hydrogels readily by absorbing water.

Preferably the alginate has a molecular weight (Mw) of 32,000 to 400,000 g/mol, more preferably 50,000 to 300,000 g/mol, especially 75,000 to 220,000 g/mol.

Derivatives of alginic acid preferably comprise alkyl groups, preferably long chain alkyl groups (e.g. dodecyl or octadecyl groups), typically formed by esterifying an aliphatic alcohol with the carboxylic acid group present in the alginate. These alginate derivatives often exhibit the typical rheological properties of physically cross-linked, gel-like networks in the semidilute regime.

Alginates typically comprise anionic polysaccharides which include a linear copolymer with homopolymeric blocks of (1-4)-linked beta-D-mannuronate (M) and its C-5 epimer alpha-L-guluronate (G) residues, respectively, covalently linked together in different sequences or blocks. The monomers can appear in homopolymeric blocks of consecutive G-residues (G-blocks), consecutive M-residues (M-blocks) or alternating M and G-residues (MG-blocks).

Alginates are highly cytocompatible and resorb slowly in vivo, and function well as an injectable material that gels in the presence of ionic moieties such as calcium and barium. Hydrogels of the present invention comprising alginate crosslinked by calcium break down slowly in vivo.

The viscosity of the hydrogel can be controlled by the molecular weight of the polymer, the concentration of calcium in the gel, or the percentage of guluronic acid (G-residues) present in the alginate polymer.

The hydrogels of the present invention preferably comprise alginate in an amount of 0.5 to 5 w/v %, more preferably 1% to 3%.

The viscosity of the hydrogel typically increases as pH decreases and reaches a maximum at about pH 3 to 3.5 as carboxylate groups in the alginate become protonated and form hydrogen bonds. Preferably the hydrogel has a pH of 5 to 10, especially 6 to 8.

Optionally the hydrogel contains one or more water-soluble organic solvents, e.g. ethanol. The amount of organic solvent present is chosen so as not to undermine hydrogel formation. Preferably, however, the hydrogel is free from organic solvents.

GDL is the cyclic ester of gluconic acid and has the following structure:

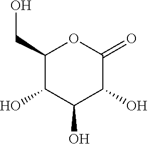 [SMILES: O=C1O[C@H](CO)[C@@H](O)[C@H](O)[C@H]1O]

When dissolved in water, the GDL undergoes hydrolysis leading to the production of gluconic acid. The gluconic acid releases calcium ions which then crosslink acidic groups in the alginate leading to the formation of a hydrogel. The amount of calcium ions released depends on a number of factors, including the concentration of the GDL in the hydrogel.

Typically, on standing in the presence of water, about 55 to 66% of the GDL hydrolysis in situ to form gluconic acid. The hydrogel preferably comprises the GDL and gluconic acid in a total amount of 0.001 to 1 w/v %, more preferably 0.05 to 0.8 and especially 0.01 to 0.5 w/v %. Thus, taking account of the fact that a part of the GDL hydrolysis in situ to form gluconic acid, the amount of GDL present in the hydrogel is preferably 0.00001 to 0.5 w/v %, more preferably 0.0005 to 0.4 w/v % and especially 0.004 to 0.2 w/v %. Furthermore, the amount of gluconic acid present in the hydrogel is preferably 0.00002 to 0.7 w/v %, more preferably 0.0005 to 0.5 w/v % and especially 0.005 to 0.4 w/v % (calculated as free acid and ignoring any calcium counter ions derived from the microparticles).

Preferably at least 75%, more preferably at least 85%, especially at least 95% and most preferably all of the GDL present in the hydrogel is dissolved in the water.

Preferably the hydrogel is sterile, as are all components of the hydrogel.

The preferred size of the microparticles depends to some extent on their intended use, e.g. the bore size of the needle which will be used to inject them into boney areas. Typically, however, the microparticles have an average diameter of 1 μm to 2,000 μm, more preferably 1 μm to 1,000 μm, especially 5 μm to 500 μm, most especially 10 μm to 100 μm. Microparticles also can be formed by crushing porous scaffolds or sponges into small parts or be formed directly by specific processes like emulsification or using a microfluidizer. Preferably the microparticles are substantially spherical (e.g. microspheres), although other shapes are possible, including egg-shaped or potato-shaped.

The hydrogel preferably comprises the microparticles in an amount of 1 to 20 w/v %, more preferably 2 to 10 w/v %, most preferably 3 to 8 w/v %. When the hydrogel is intended to be used for therapy (e.g. for repair and/or regeneration) preferably the microparticles have a mineral composition which is similar to or the same as the mineral composition of natural bone.

Preferably the microparticles have a ratio of inorganic calcium compound to recombinant gelatin of between 100:1 and 1:100, more preferably between 10:1 and 1:10 and even more preferably between 5:1 and 1:5. The most preferable ratio of the inorganic calcium compound to the recombinant gelatin is 3:2 to 2:3. By selecting the ratio of inorganic calcium compound to recombinant gelatin one may achieve good microparticle stability without sacrificing the chemical cues for bone regeneration provided by the inorganic calcium compound.

Preferably the microparticles have an average GDL content of less than 0.1 wt %, more preferably less than 0.05 wt %, especially less than 0.01 wt %, relative to the total weight of the microparticles. Most preferably the microparticles are free from GDL.

The inorganic calcium compound may optionally be added to the gelatin or formed in presence of the recombinant gelatin, for example by combining a calcium with a carbonate, sulphonate or phosphate source and allowing precipitation in the presence of the recombinant gelatin.

The inorganic calcium compound may comprise one or several calcium compounds. For example, useful calcium compounds include, without limitation, calcium carbonate, calcium sulfate, calcium lactobionate, calcium fluorite, calcium fluorophosphates, calcium chlorophosphate, calcium chloride, calcium lactate, hydroxyapatite, ceramics, calcium oxide, calcium monophosphate, calcium diphosphate, tricalcium phosphate, calcium silicate, calcium metasilicate, calcium silicide, calcium acetate, and biphasic calcium phosphate and combinations comprising two or more thereof.

The inorganic calcium compound is preferably in particulate form, e.g. in the form of crystals. The particles of inorganic calcium compound preferably have an average particles size of 1 nm to 50 μm. In contrast to homogeneous nucleation for which nucleation takes places randomly in solution, heterogeneously nucleated inorganic calcium compounds may be formed through initial association of calcium ions with carboxylic acid groups from the aspartic acid and/or glutamic acid groups of the recombinant gelatin. The resultant particles are typically crystals and these crystals may further grow in vivo within bones and thereby mimic human bone where collagen and the inorganic calcium compound are intimately linked.

The microparticles and/or the hydrogel optionally further comprise other excipients. Examples of such further excipients include synthetic and natural polymers, pharmaceutically active compounds, growth factors (e.g. bone morphogenetic proteins (BMPs), vascular endothelial growth factor (VEGF) and platelet derived growth factor (PDGF)), other proteins (e.g. follistatin), crosslinkers and natural bone components.

The inorganic calcium compound is preferably CaCO₃, especially CaCO₃ which has been obtained by precipitation or mixing of calcium carbonate in presence of the recombinant gelatin. In a preferred embodiment the microparticles are obtained by a process comprising the co-precipitation of a mixture comprising CaCO₃ and the recombinant gelatin.

Preferably the microparticles comprising an inorganic calcium compound and recombinant gelatin are in the form of recombinant gelatin microparticles containing particles of the inorganic calcium compound. For example, the inorganic calcium compound is in the form of particles and the particles of the inorganic calcium compound are distributed randomly within recombinant gelatin particles.

The amount of inorganic calcium compound present in the hydrogel is preferably 0.01 1 to 10 w/v %, more preferably 0.05 to 9 w/v %, especially 0.1 to 8.5 w/v %, more especially 0.5 to 8.4 w/v % and particularly 0.5 to 8.3 w/v %. Preferably at least a part of the calcium crosslinks the carboxy groups present in the alginate.

The amount of excipients present in the microparticles is preferably 0.0001 and 10 w/v %, more preferably 0.001 to 9 w/v %, especially 0.005 to 8 w/v %, relative to the volume of the hydrogel.

The recombinant gelatin preferably comprises at least 8% glutamic and/or aspartic acids per 60 amino acids in row with a standard deviation (SD_{ED}) of at most 8% as defined later.

The % glutamic and/or aspartic acids amount per 60 amino acids in row may be calculated by dividing the recombinant gelatin into segments, each containing 60 amino acids and, starting at the N-terminus, and disregarding the remainder, dividing the number of glutamic acid (E) and/or (preferably “and”) aspartic acid (D) residues by 60 and multiplying the resultant figure by 100%, then calculating the average for all complete rows of 60 in the recombinant gelatin. For example, in the first row of SEQ ID NO: 1 shown below there are three E's (glutamic acid residues) and three D's (aspartic acid residues) making a total of six E and D residues and ((6/60)×100=10% in total of glutamic and aspartic acid acids amount per 60 amino acids in a row (5% of E+5% of D). If one repeats this calculation for all complete rows of 60 in SEQ ID NO: 1, one achieves a figure of 9.8% GLU+ASP amount per 60 amino acids in row, as shown in Table 1 below.

Preferably the recombinant gelatin comprises at least 8% in total of glutamic acid and aspartic acids per 60 amino acids in a row, more preferably at least 8% in total of glutamic acid and aspartic acids per 60 amino acids in every complete row of 60 amino acids of the recombinant gelatin starting at the N-terminus of the recombinant gelatin.

The standard deviation (SD_{ED}) may be determined as follows: the gelatin chain is divided into segments, each containing 60 amino acids, starting at the N-terminus, and disregarding the remainder. For each of these segments the combined amount of glutamic acid (E) and aspartic acid (D) (collectively xᵢ) is determined and a standard deviation is calculated as follows:

$${SD}_{ED} = \sqrt{\frac{{\sum }_{i}^{n} {( {x}_{i} - \overline{x} )}^{2}}{( n - 1 )}} ,$$

when:
n is the total number of segments containing 60-amino acids in the gelatin; and
xᵢ is the combined amount of glutamic acid (E) and aspartic acid (D) for each segment;

$$\overline{x} = \frac{{\sum }_{i}^{n} {x}_{i}}{n}$$

Preferably the standard deviation (SD_{ED}) distribution is at most 1.30, more preferably at most 1.10.

### DESCRIPTION OF THE DRAWINGS

FIG. 1 shows the morphology of a microparticle used in the hydrogels of the present invention.

FIGS. 2a to 2e show the rheological behavior of the hydrogels under different strains and the rupture of the hydrogels.

FIGS. 3a and 3b show the thixotropic behavior (viscous state under stress and self-healing after stress is removed) of the hydrogels composed of alginate, microspheres and GDL.

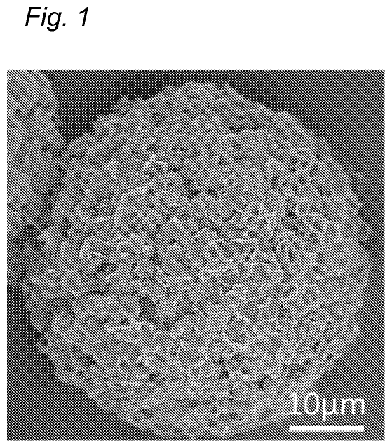

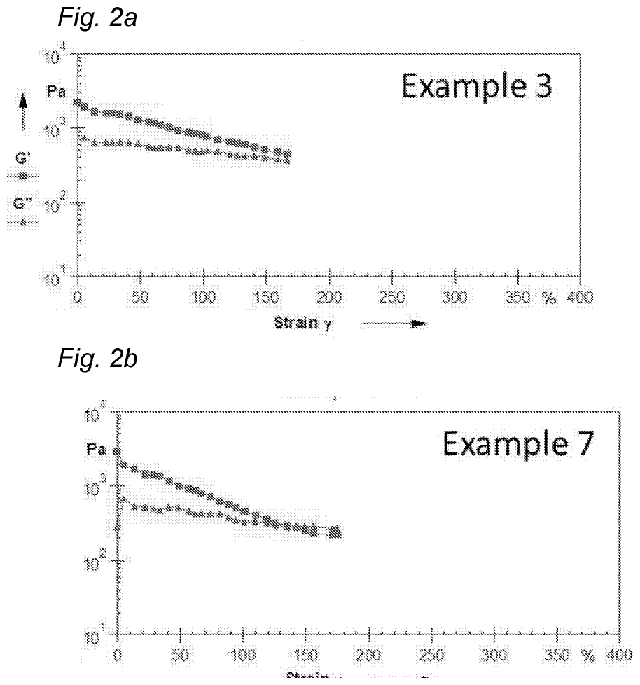

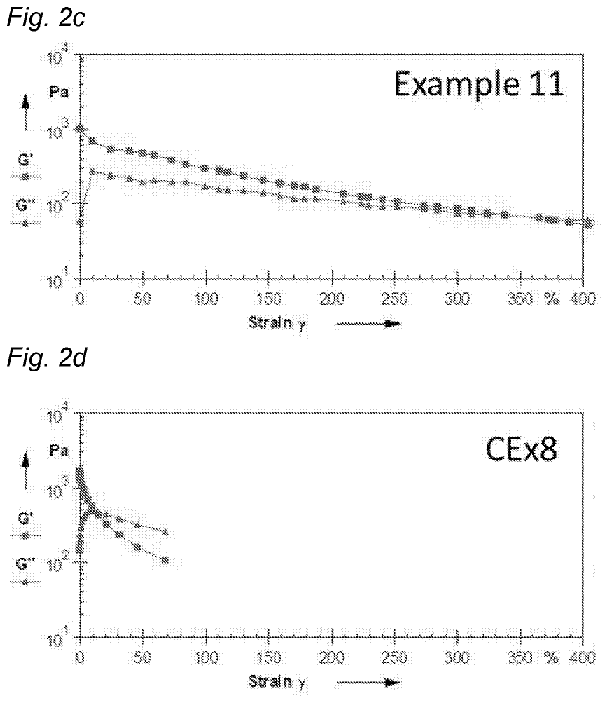

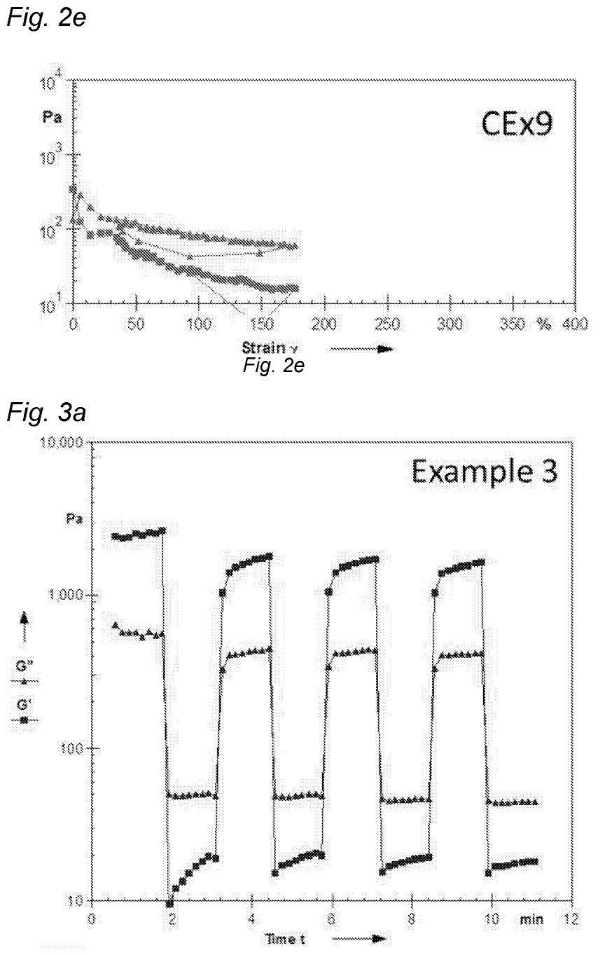

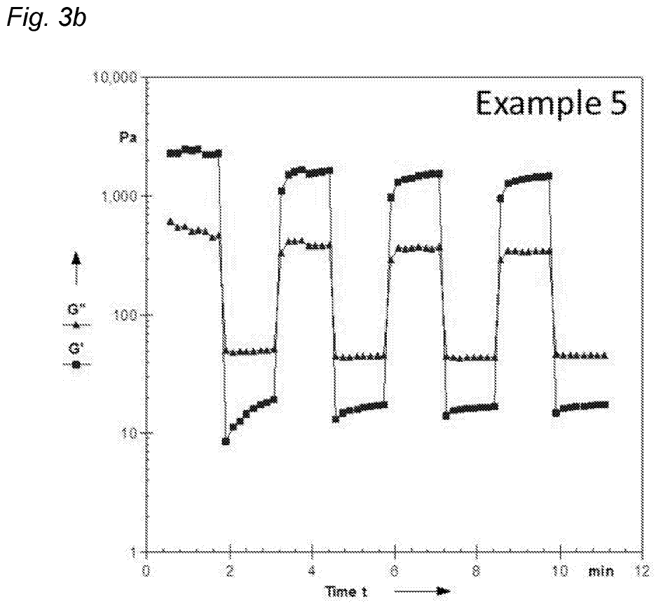

In FIGS. 2a to 2e and FIGS. 3a and 3b, G′ (square dots) is the storage (or elastic) modulus and Gii (triangular dots) is the loss (or viscous) modulus. More information is provided in the Examples section below.

The recombinant gelatin is preferably a non-fibrilar recombinant gelatin and preferably has a lower molecular weight than native gelatin. Furthermore, the recombinant gelatin preferably comprises glutamic and/or aspartic acid residues homogeneously distributed along the chain. Preferably the recombinant gelatin comprises a total amount of at least 8% glutamic and/or aspartic acids, e.g. per 60 amino acids in a row, with a standard deviation of at most 1.6. For the purpose of increasing the total calcium phosphate (or more specifically, hydroxyapatite) binding capacity, the absolute occurrence of glutamic and/or aspartic acid residues preferably is at least 9%, more preferably about 10%.

The recombinant gelatin preferably has an average molecular weight of less than 150 kDa, preferably of less than 100 kDa. Preferably the recombinant gelatin has an average molecular weight of at least 5 kDa, preferably at least 10 kDa and more preferably of at least 30 kDa. Preferred average molecular weight ranges for the recombinant gelatin include 50 kD to 100 kDa, 20 kDa to 75 kDa and 5 kDa to 40 kDa. Lower molecular weights may be preferred when higher mass concentrations of gelatins are required because of the lower viscosity.

The recombinant gelatin may be obtained commercially, e.g. from FUJIFILM under the tradename Cellnest™. The recombinant gelatin may also be prepared by known methods, for example as described in patent applications EP 0 926 543 and EP 1 014 176, the content of which is herein incorporated by reference. The methodology for preparing recombinant gelatins is also described in the publication ‘High yield secretion of recombinant gelatins by Pichia pastoris’, M.W.T. Werten et al., Yeast 15, 1087-1096 (1999). Suitable recombinant gelatins are also described in WO 2004/85473.

The amount of recombinant gelatin present in the hydrogel is preferably from 1 to 20 w/v %, more preferably 1 to 10 w/v %, especially 1.5 to 8 w/v %.

In one embodiment the recombinant gelatin comprises at least two lysine residues, said lysine residues being extreme lysine residues wherein a first extreme lysine residue is the lysine residue that is closest to the N-terminus of the gelatine and the second extreme lysine residue is the lysine residue that is closest to the C-terminus of the gelatine and said extreme lysine residues are separated by at least 25 percent of the total number of amino acids in the gelatin. Such recombinant gelatins may be obtained by, for example, the methods described in US 2009/0246282.

In another embodiment the gelatin is a the recombinant gelatin comprising at least two amino acid residues, said two amino acid residues being extreme amino acid residues, which independently are selected from an aspartic acid residue and a glutamic acid residue, wherein a first aspartic acid residue or glutamic acid residue is the aspartic acid residue or glutamic acid residue that is closest to the N-terminus of the polypeptide and the second extreme aspartic acid residue or glutamic acid residue is the aspartic acid residue or glutamic acid residue that is closest to the C-terminus of the polypeptide and said extreme aspartic acid residues and/or glutamic acid residues are separated by at least 25 percent of the total number of amino acids in the recombinant gelatin polypeptide.

In a preferred embodiment the recombinant gelatin has excellent cell attachment properties and preferably does not display any health-related risks.

The recombinant gelatin preferably has an isoelectric point of at least 5.

Preferably the recombinant gelatin is an RGD-enriched recombinant gelatin, e.g. a recombinant gelatin in which the percentage of RGD motifs related to the total number of amino acids is at least 0.4. If the RGD-enriched gelatin comprises 350 amino acids or more, each stretch of 350 amino acids preferably contains at least one RGD motif. Preferably the percentage of RGD motifs is at least 0.6, more preferably at least 0.8, more preferably at least 1.0, more preferably at least 1.2 and most preferably at least 1.5. A percentage RGD motifs of 0.4 corresponds with at least 1 RGD sequence per 250 amino acids. The number of RGD motifs is an integer, thus to meet the feature of 0.4%, a gelatin consisting of 251 amino acids should comprise at least 2 RGD sequences. Preferably the RGD-enriched recombinant gelatin comprises at least 2 RGD sequences per 250 amino acids, more preferably at least 3 RGD sequences per 250 amino acids, most preferably at least 4 RGD sequences per 250 amino acids.

The recombinant gelatin preferably comprises at least three RGD motifs. In a further embodiment an RGD-enriched gelatin comprises at least 4 RGD motifs, preferably at least 6, more preferably at least 8, even more preferably at least 12 up to and including 16 RGD motifs.

The recombinant gelatins used in this invention are preferably derived from collagenous sequences. Nucleic acid sequences encoding collagens have been generally described in the art. (See, e.g., Fuller and Boedtker (1981) Biochemistry 20: 996-1006; Sandell et al. (1984) J Biol Chem 259: 7826-34; Kohno et al. (1984) J Biol Chem 259: 13668-13673; French et al. (1985) Gene 39: 311-312; Metsaranta et al. (1991) J Biol Chem 266: 16862-16869; Metsaranta et al. (1991) Biochim Biophys Acta 1089: 241-243; Wood et al. (1987) Gene 61: 225-230; Glumoff et al. (1994) Biochim Biophys Acta 1217: 41-48; Shirai et al. (1998) Matrix Biology 17: 85-88; Tromp et al. (1988) Biochem J 253: 919-912; Kuivaniemi et al. (1988) Biochem J 252: 633640; and Ala-Kokko et al. (1989) Biochem J 260: 509-516).

Recombinant gelatins enriched in RGD motifs may also be prepared by, for example, the general methods described in US 2006/0241032.

An example of a suitable source of recombinant gelatin which may be used in the method of this invention is human COL1A1-1. A part of 250 amino acids comprising an RGD sequence is given in WO 04/85473. RGD sequences in the recombinant gelatin can adhere to specific receptors on cell surfaces called integrins.

RGD-enriched gelatins can be produced by recombinant methods described in, for example, EP-A-0926543, EP-A-1014176 or WO 01/34646, especially in the Examples of the first two mentioned patent publications. The preferred method for producing an RGD-enriched recombinant gelatin comprises starting with a natural nucleic acid sequence encoding a part of the collagen protein that includes an RGD amino acid sequence. By repeating this sequence an RGD-enriched recombinant gelatin may be obtained. Thus the recombinant gelatins can be produced by expression of nucleic acid sequence encoding such gelatins by a suitable micro-organism. The process can suitably be carried out with a fungal cell or a yeast cell. Suitably the host cell is a high expression host cells like Hansenula, Trichoderma, Aspergillus, Penicillium, Saccharomyces, Kluyveromyces, Neurospora or Pichia. Fungal and yeast cells are preferred to bacteria as they are less susceptible to improper expression of repetitive sequences. Most preferably the host will not have a high level of proteases that cleave the gelatin structure being expressed. In this respect Pichia or Hansenula offers an example of a very suitable expression system. Use of Pichia pastoris as an expression system is disclosed in EP 0 926 543 and EP 1 014 176. The microorganism may be free of active post-translational processing mechanism such as in particular hydroxylation of proline and also hydroxylation of lysine. Alternatively the host system may have an endogenic proline hydroxylation activity by which the gelatin is hydroxylated in a highly effective way.

In a further embodiment, the recombinant gelatin has less glycosylation than native gelatin, e.g. a glycosylation of less than 2 wt %, preferably less than 1 wt %, more preferably less than 0.5 wt %, especially less than 0.2 wt % and more especially less than 0.1 wt %. In a preferred embodiment the recombinant gelatin is free from glycosylation.

The degree or wt % of glycosylation refers to the total carbohydrate weight Thus the recombinant gelatins can be produced by expression of nucleic acid sequence encoding such gelatins by a suitable micro-organism. The process can suitably be carried out with a fungal cell or a yeast cell. Suitably the host cell is a high expression host cells like Hansenula, Trichoderma, Aspergillus, Penicillium, Saccharomyces, Kluyveromyces, Neurospora or Pichia. Fungal and yeast cells are preferred to bacteria as they are less susceptible to improper expression of repetitive sequences. Most preferably the host will not have a high level of proteases that cleave the gelatin structure being expressed. In this respect Pichia or Hansenula offers an example of a very suitable expression system. Use of Pichia pastoris as an expression system is disclosed in EP 0 926 543 and EP 1 014 176. The microorganism may be free of active post-translational processing mechanism such as in particular hydroxylation of proline and also hydroxylation of lysine. Alternatively the host system may have an endogenic proline hydroxylation activity by which the gelatin is hydroxylated in a highly effective way.

The degree or wt % of glycosylation preferably refers to the total carbohydrate weight per unit weight of the gelatin, as determined by, for example, MALDI-TOF-MS (Matrix Assisted Laser Desorption Ionization mass spectrometry) or by the titration method by Dubois. The term ‘glycosylation’ refers not only to monosaccharides, but also to polysaccharides, e.g. di- tri- and tetra-saccharides.

There are various methods for ensuring that glycosylation is low or absent. Glycosylation is a post-translational modification, whereby carbohydrates are covalently attached to certain amino acids of the gelatin. Thus both the amino acid sequence and the host cell (and enzymes, especially glycosyltransferases) in which the amino acid sequence is produced determine the degree of glycosylation. There are two types of glycosylation: N-glycosylation begins with linking of GlcNAc (N-actylglucosamine) to the amide group of asparagines (N or Asn) and O-glycosylation commonly links GalNAc (N-acetylgalactosamine) to the hydroxyl group of the amino acid serine (S or Ser) or threonine (T or Thr).

Glycosylation can, therefore, be controlled and especially reduced or prevented, by choosing an appropriate expression host, and/or by modifying or choosing sequences which lack consensus sites recognized by the host's glycosyltransferases. Chemical synthesis of gelatin can also be used to prepare gelatin which is free from glycosylation. Also recombinant gelatin which comprises glycosylation may be treated after production to remove all or most of the carbohydrates or non-glycosylated gelatin may be separated from glycosylated gelatin using known methods.

Surprisingly recombinant gelatins described above give rise to efficient nucleation and growth of low-crystalline inorganic calcium compound particles.

Preferably the microparticles are obtained by precipitating the inorganic calcium compound in the presence of the recombinant gelatin. For example, one may dissolve the recombinant gelatin in an aqueous solution (e.g. at a concentration typically between 1% and 30%), acidify the solution (e.g. using for example carbonic or phosphoric acid) and mixing the resultant solution with calcium hydroxide (e.g. by adding the acidified recombinant gelatin solution to a solution of calcium hydroxide). It is also possible to first mix the recombinant gelatin with a calcium source (e.g. calcium hydroxide solution) and subsequently add the carbonic or phosphoric acid. It is further possible to add the inorganic calcium compound as a fine powder to an aqueous solution of the recombinant gelatin.

After precipitating or mixing the inorganic calcium compound in the presence of the recombinant gelatin (typically allowing a crystallization process to occur in the presence of the recombinant gelatin) a composite slurry is usually obtained.

To increase the biomimetic character of the hydrogels of the present invention the Inorganic calcium compound may further comprise additives such SO₃²⁻, Na⁺, Mg²⁺, Sr²⁺, Si4⁺, Zn²⁺, SiO₄⁴⁻ and/or HPO₄²⁻ ions. In one embodiment the hydrogels of the present invention comprise one or more of such additives in a total amount of 0.01% to 25 wt %. Especially preferred are additive concentrations that mimic the amounts of such additives in natural, human bone. Preferably the gelatin is a cross-linked recombinant gelatin because this can increase the storage stability of the hydrogel. Crosslinking is preferably achieved using cross-linkable groups, e.g. carboxy or amino groups, present in the recombinant gelatin. Techniques for crosslinking gelatin are already described in literature. Mostly crosslinking occurs through the carboxylic acid or amine groups of the gelatin.

The crosslinking agent which may be used is not particularly limited. For example one may use a chemical crosslinking agent, e.g. formaldehyde, glutaraldehyde, hexamethylene diisocyanate, carbodiimides and/or cyanamide.

Preferably the crosslinking does not impair the biocompatibility of the microparticles in the hydrogel and does not generate a strong immune response. In that respect, the use of dehydrothermal treatment as a crosslinking method is preferred. Also the use of hexamethylene diisocyanate as a crosslinking agent is preferred.

In a preferred embodiment the hydrogel comprises:
a. 1 to 20 w/v % of the microparticles;
b. 0.01 to 10 w/v % of the inorganic calcium compound(s);
c. 0.01 to 1% w/v % of the GDL (or combined amount of GDL and gluconic acid); and
d. 0.5 to 5 w/v % of the alginate.

According to a second aspect of the present invention there is provided a process for preparing a hydrogel comprising the steps of precipitating an inorganic calcium compound in the presence of a recombinant gelatin to form microparticles comprising the inorganic calcium compound and the recombinant gelatin and mixing the so formed microparticles with a composition comprising water, alginate and GDL.

The process preferably further comprises hydrolysing at least a part of the GDL to form gluconic acid, preferably by exposing the GDL to biological conditions of a human or animal body.

According to a third aspect of the present invention there is provided a process for preparing a hydrogel comprising injecting into a human or animal body a composition comprising:

water;

an alginate;

a glucono-delta-lactone (GDL); and

microparticles comprising an inorganic calcium compound and recombinant gelatin.

In the second and third aspects of the present invention the preferred inorganic calcium compound, recombinant gelatin, GDL and alginate are as described herein in relation to the first aspect of the present invention.

According to a fourth aspect of the present invention there is provided a medicament comprising a sealed bottle or ampoule and a hydrogel according to the first aspect of the present invention, wherein the hydrogel is present in the sealed bottle or ampoule.

The invention will now be illustrated with the following non-limiting Examples.

### EXAMPLES

### Materials:

| Material | Source |
|---|---|
| Hexamethylene diisocyanide | Sigma-Aldrich (St. Louis, MO, USA) |
| (HMDIC) (>98.0% purity) | |
| Corn Oil | |
| Sterile Sodium Chloride | |
| Calcium Carbonate (CaCO₃) | |
| fine powder (average | |
| particle size < 1 μm) | |
| GDL | |
| Ethanol | Millipore (Billerica, MA, USA) |
| Acetone | |
| Hydrochloric Acid | |
| Pronova SLM 20 (sterile | Novamatrix (Sandvika, Norway) |
| alginate where over 50% of | |
| the monomer units are | |
| mannuronate) | |
| Pronova SLG 20 (sterile | |
| alginate where over 60% of | |
| the monomer units are | |
| guluronate) | |
| rhBMP-2% | A 2 wt % solution of mature re- |
| | combinant human bone morphogenetic |
| | protein-2 (rhBMP-2 peptide) |
| | containing amino acids of BMP-2 |
| | 283 to 396 plus an N-terminal Met- |
| | Ala. Expressed in E. coli, isolated |
| | from inclusion bodies, renatured |
| | and purified as described by |
| | Kirsch T, Nickel J, Sebald W |
| | (Isolation of recombinant BMP |
| | receptor IA ectodomain and its |
| | 2:1 complex with BMP-2. FEBS |
| | letters. 2000; 468: 215-9) |
| Gel | Type A native pigskin-derived gelatin |
| | (i.e. not recombinant and therefore |
| | outside the scope of the present |
| | claims). |

### Preparation of Recombinant Gelatins

Recombinant gelatins (SEQ ID NO: 1 and 2) were prepared based on a nucleic acid sequence that encodes for a part of the gelatin amino acid sequence of human COLIAI-I and modifying this nucleic acid sequence using the methods disclosed in EP-A-0926543, EP-A-1014176 and WO01/34646. The gelatins did not contain hydroxyproline and comprised the amino acid sequences identified herein as in SEQ ID NO: 1 or 2. Except for the last incomplete row, the total amount of GLU+ASP per row of 60 amino acids is shown on the right side of each row.

| | number of |
|---|---|
| | (GLU+ASP) |
| | residues |
| | per 60 |
| | amino |
| | acids in |
| | a row: |
| SEQ ID NO: 1 | |
| GAPGAPGLQGAPGLQGMPGERGADGLPGPKGERGDAGPKG | 6 |
| ADGAPGAPGLQGMPGERGAA | |
| GLPGPKGERGDAGPKGAAGAPGKDGVRGLAGPIGPPGERG | 7 |
| AAGLPGPKGERGDAGPKGAD | |
| GAPGKDGVRGLAGPIGPPGPAGAPGAPGLQGMPGERGAAG | 5 |
| LPGPKGERGDAGPKGADGAP | |
| GKDGVRGLAGPPGAPGLQGAPGLQGMPGERGAAGLPGPKG | 5 |
| ERGDAGPKGADGAPGAPGLQ | |
| GMPGERGAAGLPGPKGERGDAGPKGAAGAPGKDGVRGLAG | 6 |
| PIGPPGERGAAGLPGPKGER | |
| GDAGPKGADGAPGKDGVRGLAGPIGPPGPAGAPGAPGLQG | 6 |
| MPGERGAAGLPGPKGERGDA | |
| GPKGADGAPGKDGVRGLAGPPGAPGLQGAPGLQGMPGERG | 6 |
| AAGLPGPKGERGDAGPKGAD | |
| GAPGAPGLQGMPGERGAAGLPGPKGERGDAGPKGADGAPG | 6 |
| KDGVRGLAGPIGPPGERGAA | |
| GLPGPKGERGDAGPKGADGAPGKDGVRGLAGPIGPPGPAG | 6 |
| APGAPGLQGMPGERGADGLP | |
| GPKGERGDAGPKGADGAPGKDGVRGLAGPPG | |
| SEQ ID NO: 2 | |
| GAPGAPGLQGAPGLQGMPGERGAAGLPGPKGERGDAGPKG | 4 |
| AAGAPGAPGLQGMPGERGAA | |
| GLPGPKGERGDAGPKGAAGAPGKDGVRGLAGPIGPPGERG | 6 |
| AAGLPGPKGERGDAGPKGAA | |
| GAPGKDGVRGLAGPIGPPGPAGAPGAPGLQGMPGARGADG | 5 |
| LPGPKGERGDAGPKGADGAP | |
| GKAGVRGLAGPPGAPGLQGAPGLQGMPGARGAAGLPGPKG | 1 |
| ARGDAGPKGAAGAPGAPGLQ | |
| GMPGERGAAGLPGPKGERGDAGPKGAAGAPGKDGVRGLAG | 6 |
| PIGPPGERGAAGLPGPKGER | |
| GDAGPKGADGAPGKDGVRGLAGPIGPPGPAGAPGAPGLQG | 6 |
| MPGERGAAGLPGPKGERGDA | |
| GPKGADGAPGKDGVRGLAGPPGAPGLQGAPGLQGMPGERG | 5 |
| AAGLPGPKGARGDAGPKGAD | |
| GAPGAPGLQGMPGARGAAGLPGPKGERGDAGPKGADGAPG | 5 |
| KDGVRGLAGPIGPPGERGAA | |
| GLPGPKGERGDAGPKGAAGAPGKAGVRGLAGPIGPPGPAG | 3 |
| APGAPGLQGMPGERGAAGLP | |
| GPKGERGDAGPKGADGAPGKDGVRGLAGPPG | |

The distribution of GLU+ASP in the gelatin is represented by the standard deviation of the amounts per row (see Table 1 below).

SEQ ID NO: 1 and 2 were used to prepare the various hydrogels described in the Examples below.

TABLE 1
Amount/distribution of (GLU + ASP) in SEQ ID NO: 1 and 2.

| | | Average Number of | Standard deviation |
|---|---|---|---|
| | | (GLU + ASP) | (GLU + ASP) amount |
| | % Amount of | residues per 60 | per 60 amino acids in |
| gelatin | (GLU + ASP) | amino acids in row | row |
| SEQ ID NO: 1 | 9.8 | 5.9 | 0.60 |
| SEQ ID NO: 2 | 9.8 | 5.9 | 1.69 |

### Step 1) Preparation of Microparticles by Precipitation of CaCO₃ in the Presence of Gelatins.

A 20% aqueous (20 g of gelatins SEQ ID NO: 1 or NO: 2 or Type A pigskin derived gelatin) solution was prepared and mixed with CaCO₃ fine powder (with a size of <1 μm) in a 1:1 (w/w) ratio of gelatin to CaCO₃. This suspension was emulsified in corn oil at 50° C. while stirring the emulsion at 800 rpm for 20 min. After cooling down the emulsion, the emulsified microparticles were washed three times with acetone. After overnight drying at 60° C., microparticles were sieved to 50-72 μm size using sieves (Retsch GmbH, Haan, Germany). Particles were crosslinked for each gelatin using hexamethylene diisocyanide (HMDIC) or dehydrothermal crosslinking (DHT), as indicated in Table 2 below.

Crosslinking with HMDIC: 1 g of spheres and 1 mL of HMDIC (>98.0% pure, Sigma)) were mixed in 100 ml ethanol for 1 day. Excess cross-linker was removed by washing several times with ethanol.
Crosslinking by DHT: 1 g of spheres were crosslinked at 160° C. in vacuum (˜5.10⁻³ mbar) oven for 4 days.

In Comparative Example 10, the inorganic calcium compound (CaCO₃) was removed from the microparticles by treating the microparticles with excess hydrochloric acid (1M, Merck) until carbon dioxide formation stopped. The calcium-free microparticles were then washed 3 times with deionised water and subsequently dried overnight drying at 60° C.

All crosslinked particles were gamma sterilized afterwards by Synergy Health (Etten Leur, The Netherlands) prior to use in in vitro and in vivo experiments.

Step 2) Loading of the Microparticles with Excipients

Microparticles prepared in step 1 (68 mg) were incubated with 170 μL rhBMP-2 at a concentration of 122.5 μg/mL at 4° C. overnight.

For Examples 13, 14 and CEx7, 136 mg were incubated with 170 μL rhBMP-2 at a concentration of 122.5 μg/mL.

### Step 3) Preparation of the Hydrogels

Two alginate solutions were prepared based on alginate SLM20 or SLG20 by adding 0.9% sterile sodium chloride to create 2% w/v alginate (SLM or SLG) solutions.

The microparticles from step 2 were added to 1014 μL of 2% w/v of alginate SLM20 or 1014 μL of 2% w/v alginate SLG20. Immediately 106 μL of 0.06M fresh glucono delta lactone (GDL) solution was added and mixed.

Also Comparative Examples CEx8 and CEx9 were prepared in which the microparticles and BMP-2 were omitted. When required additional 0.9% sterile sodium chloride was used to ensure that the Comparative Examples had the same alginate concentration as the actual Examples.

Comparative Example CEx8 was prepared mixing 1014 μL of 2% w/v SLM with 276 μL of 0.25% calcium chloride.

Comparative Example CEx9 was prepared by mixing 34 mg CaCO₃, 170 μL 0.9% sodium chloride, 1014 μL of 2% w/v alginate SLM20 and 106 μL of 0.06M fresh glucono delta lactone (GDL).

Comparative Example CEx10 was prepared by adding the microparticles from step 1 without CaCO₃ to 1014 μL of 2% w/v of alginate SLM20 and mixing 34 mg CaCO₃, and 106 μL of 0.06M fresh glucono delta lactone (GDL).

The resultant hydrogels were as described in Table 2 below.

In Table 2:
SLM means Pronova SLM 20 (an alginate) and % is the w/v %, i.e. the weight of alginate relative to the total volume of the composition
SLG means Pronova SLG 20 (an alginate) and % is the w/v %, i.e. the weight of alginate relative to the total volume of the composition
MS % means the w/v % (i.e. the weight of microparticles relative to the total volume of the composition) of the relevant microparticle derived from the Gelatin (Gel) and CaCO₃ indicated in the next two columns of Table 2;
Gel ID/% shows the gelatin used (SEQ1=SEQ ID NO: 1, SEQ2=SEQ ID NO: 2; and Gel=Type A pigskin (Comparative).

TABLE 2
Hydrogels and Comparative Hydrogels Showing the Percentage (w/v) of each Component

| | Alginate | | | | | | |
|---|---|---|---|---|---|---|---|
| Example | ID/% | MS % | Gel ID/(%) | CaCO₃% | GDL % | rhBMP-2 % | Crosslinking MS |
| 1 | SLM/1.5 | 5.2 | SEQ1/2.6% | 2.6% | 0.089% | 0.0016% | HMDIC |
| 2 | SLM/1.5 | 5.2 | SEQ2/2.6% | 2.6% | 0.089% | 0.0016% | HMDIC |
| CEx1 | SLM/1.5 | 5.2 | Gel/2.6% | 2.6% | 0.089% | 0.0016% | HMDIC |
| 3 | SLG/1.5 | 5.2 | SEQ1/2.6% | 2.6% | 0.089% | 0.0016% | HMDIC |
| 4 | SLG/1.5 | 5.2 | SEQ2/2.6% | 2.6% | 0.089% | 0.0016% | HMDIC |
| CEx2 | SLG/1.5 | 5.2 | Gel/2.6% | 2.6% | 0.089% | 0.0016% | HMDIC |
| 5 | SLM/1.5 | 5.2 | SEQ1/2.6% | 2.6% | — | 0.0016% | HMDIC |
| 6 | SLM/1.5 | 5.2 | SEQ2/2.6% | 2.6% | — | 0.0016% | HMDIC |
| CEx3 | SLM/1.5 | 5.2 | Gel/2.6% | 2.6% | — | 0.0016% | HMDIC |
| 7 | SLG/1.5 | 5.2 | SEQ1/2.6% | 2.6% | — | 0.0016% | HMDIC |
| 8 | SLG/1.5 | 5.2 | SEQ2/2.6% | 2.6% | — | 0.0016% | HMDIC |
| CEx4 | SLG/1.5 | 5.2 | Gel/2.6% | 2.6% | — | 0.0016% | HMDIC |
| 9 | SLM/1.5 | 5.2 | SEQ1/2.6% | 2.6% | — | 0.0016% | DHT |
| 10 | SLM/1.5 | 5.2 | SEQ2/2.6% | 2.6% | — | 0.0016% | DHT |
| CEx5 | SLM/1.5 | 5.2 | Gel/2.6% | 2.6% | — | 0.0016% | DHT |
| 11 | SLM/1.5 | 5.2 | SEQ1/2.6% | 2.6% | 0.089% | 0.0016% | DHT |
| 12 | SLM/1.5 | 5.2 | SEQ2/2.6% | 2.6% | 0.089% | 0.0016% | DHT |
| CEx6 | SLM/1.5 | 5.2 | Gel/2.6% | 2.6% | 0.089% | 0.0016% | DHT |
| 13 | SLG/1.5 | 10.4 | SEQ1/2.6% | 5.2% | 0.089% | 0.0016% | HMDIC |
| 14 | SLG/1.5 | 10.4 | SEQ2/2.6% | 5.2% | 0.089% | 0.0016% | HMDIC |
| CEx7 | SLG/1.5 | 10.4 | Gel/2.6% | 5.2% | 0.089% | 0.0016% | HMDIC |
| CEx8 | SLM/1.5 | — | — | — | — | — | — |
| CEx9 | SLM/1.5 | — | — | 2.6% | 0.089% | — | — |
| CEx10 | SLM/1.5 | 2.6 | SEQ1/2.6% | 2.6%* | 0.089% | 0.0016% | HMDIC |

*in CEx10 the CaCO3 was removed_from the microparticles by treating the microparticles with excess hydrochloric acid, as described above.

### Evaluation of the Hydrogels and Comparative Hydrogels Described in Table 2

The mechanical properties of prepared hydrogels were measured by a Rheometer (Anton Paar MCR301, Graz, Austria). A 20 mm diameter parallel plate measuring system was used. After sample addition to the plate, silicon oil was applied to the edges to prevent evaporation. Storage (or elastic) modulus (G′) and loss (or viscous) modulus (G″) were measured between 0%-400% strain at 37° C. to assess the viscoelastic region. To study the thixotropic behaviour, a different setting was used which included two-step repeating cycle. At the first step of the cycle, storage and loss moduli were measured at 1% strain, at 1 Hz, at 37° C. At the second step, 500% strain, 1 Hz frequency, 37° C. temperature was applied. The cycle was repeated several times to characterize thixotropic behavior. Normal force was set to 0.1 N. Strain-dependent oscillatory rheology of gelatin microparticle alginate hydrogels, as shown in Table 3, showed an extremely broad linear viscoelastic region in addition to network rupture at high strains at 150% for Example 7, Example 8 and Comparative Example 4 hydrogels. The mechanical properties were increased with addition of GDL that rupture occurs for Examples 1 to 4 and Comparative Examples CEx1 and CEx2 at a strain of >170% (Table 3). This showed the importance of GDL in the composition. The highest strain for network rupture was observed for formulations Examples 11 and 12 and

Comparative Example CEx6 which contained DHT crosslinked microparticles shows a network rupture at very high strain (>350), indicated that composites containing DHT crosslinked microparticles have higher mechanical properties.

The Comparative Example CEx8 hydrogel (without microparticles) broke at about 12% strain. In Comparative Example CEx9 the hydrogel did not contain microparticles but contained 1 μm CaCO₃ crystals and broke at about 40% strain. Also Comparative Example CEx10 in which the microparticles were free from inorganic calcium compounds (the calcium compounds were removed as described above) and to which 34 mg CaCO₃ had been added had a low rupture strain of only 35%. The results shown in Table 3 show the importance of the hydrogels of the invention having the claimed features.

TABLE 3

| | Strain % at which |
|---|---|
| Example | the structure breaks |
| Comparative Examples CEx8, CEx9 | 12-40 |
| and CEx10 | |
| Examples 9 to 12 and Comparative | >350 |
| Examples CEx5 and CEx6 | |
| Examples 1 to 4 and Comparative | >170 |
| Examples CEx1 and CEx2 | |
| Examples 7 and 8 and Comparative | 150 |
| Example CEx4 | |

The hydrogels of Examples 1 to 8 and Comparative Examples CEx1 to CEx4 and Examples 13 and 14 and Comparative Example CEx7 possessed self-thinning behaviour under stress and self-recovery of the hydrogel after the stress had been removed. This behaviour proves that the gel will be reformed in situ directly after injection in vivo. The formed hydrogels showed good mechanical properties as it could be seen from their storage (or elastic) moduli. When stress was removed, the storage moduli were between 1-2 kDa in both alginate gel formulations which were comparable to that of endothelial tissue and stromal tissue.

### Cell Attachment on In Situ Gelling Hydrogels

C2C12 cells (muscle fibroblast mouse cells CRL-1772 from ATCC) were cultured at 37° C. and 5% CO₂ in DMEM (Dulbecco's modified eagle's medium from Invitrogen) media supplemented with 10% fetal bovine serum (FBS) (Sigma) and 1% penicillin-streptomycin (Sigma).HG compositions were prepared as described above. From these formulations, 200 μL of was added to each well of 24-well-plates.

C2C12 cells were seeded on hydrogels as 4750 cells/well. After 5 days of cell seeding, cells were stained with Live/Dead (Invitrogen) mixture for approximately 45 min. After staining, cells were visualized under fluorescent light by Olympus BX60 light microscope. The results by visual inspection of the samples (see table 4) show that cells preferably attached to microparticles inside the hydrogel formulation rather than only gel. Further it is shown that RGD containing recombinant gelatin having enhance cell attachment in vitro.

TABLE 4
cell attachment by visual inspection of C2C12 cells

| | Visual inspection |
|---|---|
| 1 | ++ |
| 2 | ++ |
| CEx1 | + |
| 3 | ++ |
| 4 | ++ |
| CEx2 | + |
| 5 | ++ |
| 6 | ++ |
| CEx3 | + |
| 7 | ++ |
| 8 | ++ |
| CEx4 | + |
| 9 | ++ |
| 10 | ++ |
| CEx5 | + |
| 11 | ++ |
| 12 | ++ |
| CEx6 | + |
| 13 | +++ |
| 14 | +++ |
| CEx7 | + |
| CEx8 | − |
| CEx9 | − |
| CEx10 | ++ |

+++is best and −is worst

1. A hydrogel comprising:
water;
an alginate;
a glucono-delta-lactone (GDL); and
microparticles comprising an inorganic calcium compound and recombinant gelatin.

2.-10. (canceled)

11. The hydrogel according claim 1 which becomes fluid when agitated and resolidifies when resting.

12. The hydrogel according to claim 1 comprising:
a. 1 to 20 w/v % of the microparticles;
b. 0.01 to 10 w/v % of inorganic calcium compound(s);
c. 0.01 to 1% w/v % of the GDL; and
d. 0.5 to 5 w/v % of the alginate.

13.-19. (canceled)

20. The hydrogel according to claim 1 wherein the gelatin is a recombinant gelatin cross-linked with formaldehyde, glutaraldehyde, hexamethylene diisocyanate, a carbodiimide, cyanamide and/or by dehydrothermal treatment.

21.-23. (canceled)

24. The hydrogel according to claim 1 wherein the microparticles have an average diameter of 1 μm to 1000 μm.

25.-27. (canceled)

28. The hydrogel according to claim 1 wherein the weight ratio of inorganic calcium compound to the recombinant gelatin is between 3:2 to 2:3.

29. (canceled)

30. The hydrogel according to claim 1 which further comprises one or more of a synthetic polymers, natural polymers, pharmaceutically active compounds, growth factors, proteins, crosslinkers or natural bone components.

31. The hydrogel according to claim 1 wherein the gelatin comprises glutamic acid and aspartic acid and the standard deviation (SD_{ED}) distribution of the combined amounts of glutamic acid and aspartic acid in each row of 60 amino acids of said recombinant gelatin is at most 1.30.

32. The hydrogel according to claim 1 which is an injectable hydrogel.

33.-34. (canceled)

35. The hydrogel according claim 1 wherein the inorganic calcium compound is in the form of particles and the particles of the inorganic calcium compound are distributed randomly within recombinant gelatin particles.

36.-39. (canceled)

40. A process for preparing a hydrogel comprising the steps of precipitating an inorganic calcium compound in the presence of a recombinant gelatin to form microparticles comprising the inorganic calcium compound and the recombinant gelatin and mixing the so formed microparticles with a composition comprising water, alginate and GDL.

41. The process according to claim 48 which further comprises the step of hydrolysing at least a part of the GDL to form gluconic acid.

42. The medicament comprising a sealed bottle or ampoule and a hydrogel according to claim 1 wherein the hydrogel is present in the sealed bottle or ampoule.

43. The hydrogel according to claim 1 comprising:
a. 1 to 20 w/v % of the microparticles;
b. 0.01 to 10 w/v % of inorganic calcium compound(s);
c. 0.01 to 1% w/v % of the GDL; and
d. 0.5 to 5 w/v % of the alginate;
wherein the gelatin comprises amino acids and at least 8% of each set of 60 amino acids of the recombinant gelatin are glutamic acid and/or aspartic acid.

44. The hydrogel according to claim 43 wherein the gelatin is a recombinant gelatin cross-linked with formaldehyde, glutaraldehyde, hexamethylene diisocyanate, a carbodiimide, cyanamide and/or by dehydrothermal treatment.

45. The hydrogel according to claim 43 wherein the weight ratio of inorganic calcium compound to the recombinant gelatin is between 3:2 to 2:3.

46. The hydrogel according to claim 43 wherein the gelatin comprises glutamic acid and aspartic acid and the standard deviation (SD_{ED}) distribution of the combined amounts of glutamic acid and aspartic acid in each row of 60 amino acids of said recombinant gelatin is at most 1.30.

47. The hydrogel according to claim 43 wherein:
(i) the gelatin is a recombinant gelatin cross-linked with formaldehyde, glutaraldehyde, hexamethylene diisocyanate, a carbodiimide, cyanamide and/or by dehydrothermal treatment;
(ii) the weight ratio of inorganic calcium compound(s) to the recombinant gelatin is between 3:2 to 2:3; and
(iii) the gelatin comprises glutamic acid and aspartic acid and the standard deviation (SD_{ED}) distribution of the combined amounts of glutamic acid and aspartic acid in each row of 60 amino acids of said recombinant gelatin is at most 1.30.

48. The hydrogel according to claim 47 wherein the microparticles have an average diameter of 1 μm to 1000 μm.
